(19) United States
(12) Patent Application Publication
(10) Pub. No.: US 2013/0089185 A1
(43) Pub. Date: Apr. 11, 2013

(54) DENTAL X-RAY RECEPTOR POSITIONING DEVICE

(71) Applicants: William WINTERS, New Rochelle, NY (US); Peter MITROPOULOS, New York, NY (US); Joel GLICKMAN, Jupiter, FL (US); George SMITH, Perkasie, PA (US); Charles RODGERS, Langhorne, PA (US)

(72) Inventors: William WINTERS, New Rochelle, NY (US); Peter MITROPOULOS, New York, NY (US); Joel GLICKMAN, Jupiter, FL (US); George SMITH, Perkasie, PA (US); Charles RODGERS, Langhorne, PA (US)

(21) Appl. No.: 13/268,022

(22) Filed: Oct. 7, 2011

(51) Int. Cl. A61B 6/14

(52) U.S. Cl. 378/170

(57) ABSTRACT

A dental X-ray positioning device includes a bite block for gripping receptor. The bit block may be adjusted to accept receptors of various dimensions. The bite block provides the clinician with greater freedom to accurately position the receptor relative to the X-ray source without having to utilize many different devices. The system thereby improves efficiency during X-ray exams and it also guarantees parallel placement of the receptor relative to the X-ray source and to the dentition being examined. The system also incorporates two soft “jaws” that during use prevent the receptor from drooping or failing to remain in parallel position relative to the bite block, the X-ray source, and/or the dentition.

### RELATED APPLICATIONS

None

### BACKGROUND OF THE INVENTION

A. Field of Invention

This invention pertains to a device positioning and holding X-ray sensitive receptors used for recording X-ray images in the dental field.

B. Description of the Prior Art

Dental radiographs are made using various types of sensors or image “receptors.” (In this application, these two terms are used interchangeably). These receptors are placed behind the dentition and then an image is generated by directing an X-ray beam towards the dentition. Receptors commonly in use today include film, CCD- or CMOS-type sensors or phosphor plates. A common practice is to use some type of alignment device that places the image receptor in parallel alignment to the patient's dentition as well as to the source of the X-rays. This parallel alignment is critical to obtaining an accurate X-ray and for proper diagnosis. Without such a device, the receptor may not be aligned properly to the X-ray source. As a result, the image may be “cone cut”, i.e., part of the receptor may fail to be within the field of the X-ray source, or the dentition may not be normal to the receptor and to the X-ray source, which would result in a distorted image (e.g., elongation or foreshortening). A distorted dentition image is undesirable because it hinders proper diagnosis and treatment. Moreover, when a distorted image is obtained, frequently it may have to be retaken, thereby exposing the patient to unnecessary radiation and requiring additional expensive and time consuming activity by a technician. These types of devices are found in U.S. Pat. No. 6,343,875, U.S. Pat. No. 3,473,026 and U.S. Des. 237,016, all incorporated herein by reference.

The use of digital sensors, either CCD- or CMOS-based receptors, have become fairly common in the art. These sensors are made of various sizes. The height of the sensors varies as does the thickness. Many sensors incorporate a wire that connects the sensor to a computer for image downloading. These wires may be attached to the sensor in the middle of the back of the sensor, along the short side, or anywhere in-between.

Presently, dentists use a basket attached to a bite block which is designed for a specific sensor size. A different basket is available for each block size. As an alternative option, the dentist can use a bite block that has a plate with an adhesive backing, so the sensor can be affixed via this adhesive to the bite block. With this prior art product, the adhesive can only be used one time per patient, and thereafter the dentist may not re-use the bite block. Also, since the adhesion is actually taking place between the bite block and an outer protective barrier bag holding the sensor (to prevent cross contamination between patients), the sensor may sag within the bag, thus taking the sensor out of parallel alignment to the dentition as well as the X-ray source.

A need exists, therefore, for a single sensor holder/bite block assembly or apparatus which will accommodate different sizes and shapes of X-ray sensors and accordingly is re-useable for several patients.

### OBJECTIVES AND SUMMARY OF THE INVENTION

It is an object of the invention to provide an improved dental X-ray bite block for sensors.

It is another object of the invention to provide a bite block that incorporates a locking hinge mechanism so as to adjust to various sizes of sensors quickly and easily.

It is a further object of this invention to provide a bite block and sensor holder assembly which can be employed to assure consistent and proper parallel alignment between the sensor, the X-ray source, and the dentition of the patient.

It is yet another object of this invention to provide such a device that is made to be autoclaved and re-used to prevent cross contamination between patients.

It is yet another object of this invention that the adjustable sensor holders are made in different sizes and are each color coded for easy identification, making sensor positioning quick, accurate, and easy for the practitioner.

The present invention pertains to an X-ray receptor positioning device for use during dental X-ray procurement procedures. More particularly, the invention includes a modular bite block constructed and arranged to receive and hold any commercially X-ray receptor. The bite block includes two arms that can be positioned at multiple discrete spacings from each other, said spacings being selected to accommodate the various receptors between the arms. A locking hinge mechanism is used to lock the arms in place while an X-ray image is being recorded by the receptor. The arms are preferably sized and constructed to hold the receptors in either a vertical or a horizontal position.

The bite block also provides a biting surface for the patient and includes an exemplary guide arm and positioning ring so as to align the source of the X-rays with the media and the dentition as well as assuring all elements are in proper alignment, thus preventing either elongation or foreshortening of the image which may have an impact on a correct diagnosis.

### BRIEF DESCRIPTION OF THE DRAWINGS

FIG. 1 shows an orthogonal side view of a bite block constructed in accordance with this invention;

FIG. 2 shows another orthogonal view of the bite block;

FIG. 3 shows an orthogonal view of the bite block loaded with a receptor;

FIG. 4 shows a side exploded view of the bite block;

FIG. 5 shows a side view of the bite block with two inserts;

FIG. 6 shows a side view of the bite block with the inserts installed;

FIG. 7 shows an orthogonal view of the bite block assembled with an aligning device;

FIG. 8 shows a side view of the bite block and aligning device;

FIG. 9 shows a plan view of member 12;

FIG. 10 shows a bottom view of member 12;

FIG. 11 shows a side view of FIG. 12;

FIG. 12 shows a partial enlarged bottom view of a tongue on member 12;

FIG. 13 shows an end view of member 12;

FIG. 14 shows a side view of member 14;

FIG. 15 shows a plan view of member 14; and

FIG. 16 shows the bite block formed by joining members 12 and 14.

### PREFERRED EMBODIMENTS FOR CARRYING OUT THE INVENTION

An X-ray receptor positioning device according to the invention includes a bite block that holds an appropriate receptor (e.g., film, phosphor plate, CCD or CMOS type sensor) so that the receptor is parallel to the bite plane. The bite block is attached to a guide assembly that includes a guide arm and a positioning ring. Guide assemblies of this type are disclosed for example in U.S. Pat. No. 3,473,026 and U.S. Des. 237,016.

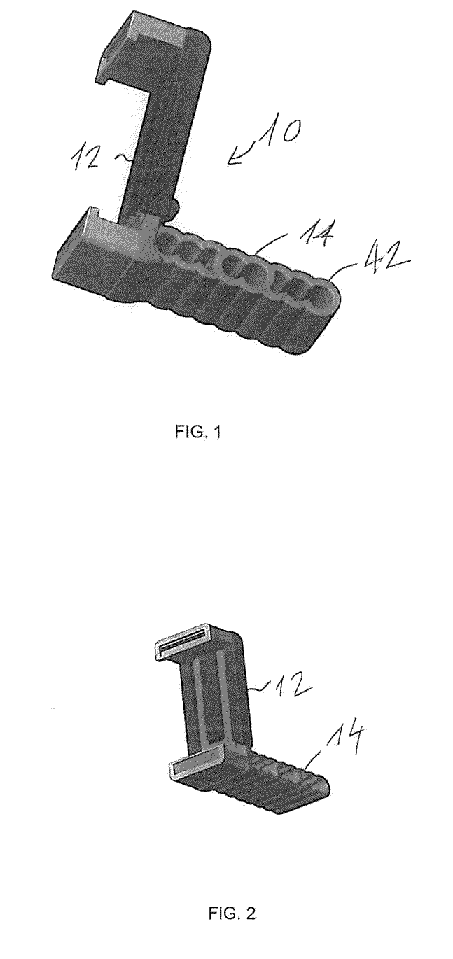

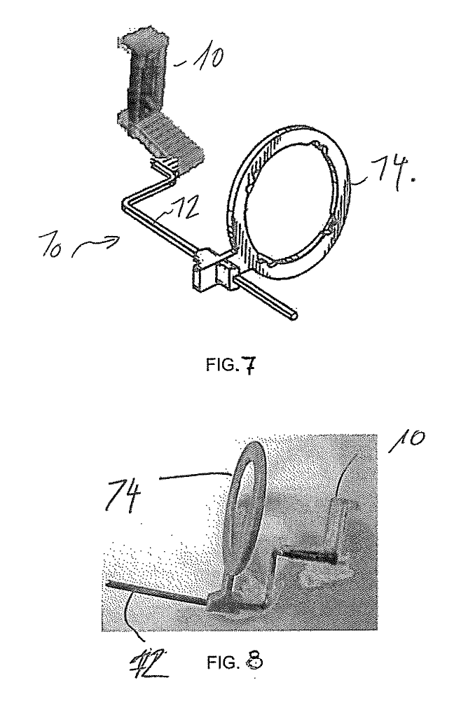

Details of the bite block are shown in FIGS. 1-6, and 9-16.

The bite block is sized and shaped to fit in the anterior and posterior regions of the mouth of a person and may also be designed so that the receptor is positioned half way to the bite block for the purpose of taking a “bite wing” exposure. The bite block assembly can be placed in an autoclave for sterilization. Moreover, since the X-ray beam has to pass through the bite block, it must be made of a material that is transparent to X-rays. The bite block may be made from several materials approved for this type of process, such as a glass-filled nylon, Fortron, Vydyne and Vectra LCP.

Bite block 10 includes two generally L-shaped members 12 and 14 (see FIG. 4). Member 12 is formed of an elongated member 16 having an inner surface 18 and an outer surface 20. At the lower end 22 of member 16, there is provided an actuating element or button 24. At the end 26 opposite button 24 there is an extension 28. Extension 28 terminates with a lip 30 turned downwardly.

Details of member 12 are shown in FIGS. 9-13.

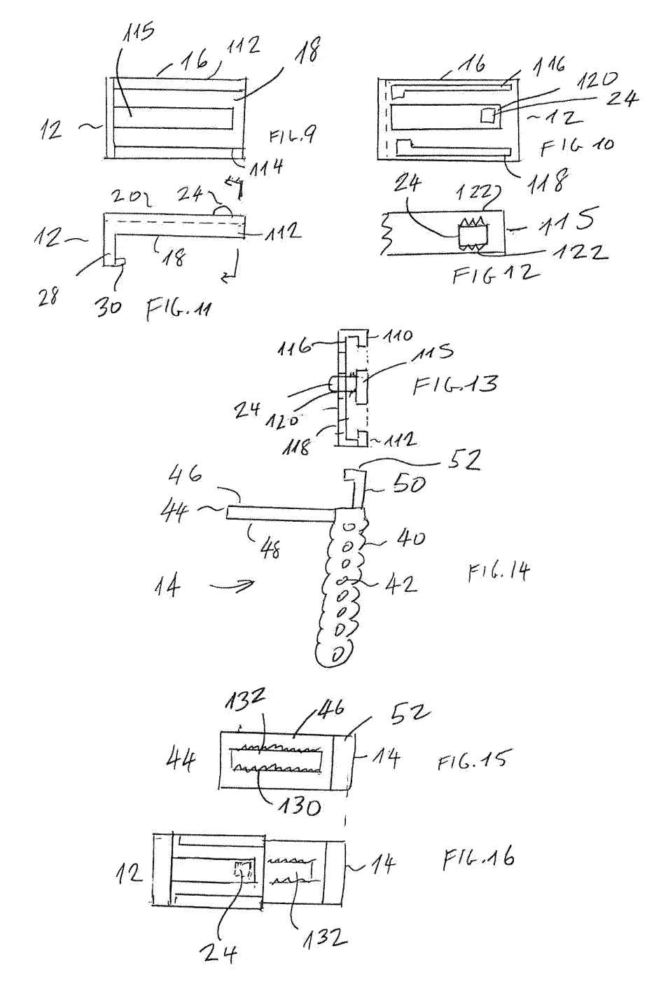

Inner surface 18 is formed with two lateral L-shaped rails 112, 114 (seen in detail in FIG. 13) facing each other to form a track. The inner surface 18 is further provided with an elongated tongue 115. Tongue 115 is cantilevered and is provided with button 24 at one end.

Three longitudinal channels are provided in member 12, two of which (116, 118) being disposed under the rails 112, 114 and one, 120, in the middle. As can be seen in FIGS. 11, 13, button 24 is sized and shaped so that it extends through the channel 120 with its tip being accessible from outer surface 20. Button 24 is provided on two of its sides with teeth 122, as shown in FIG. 12.

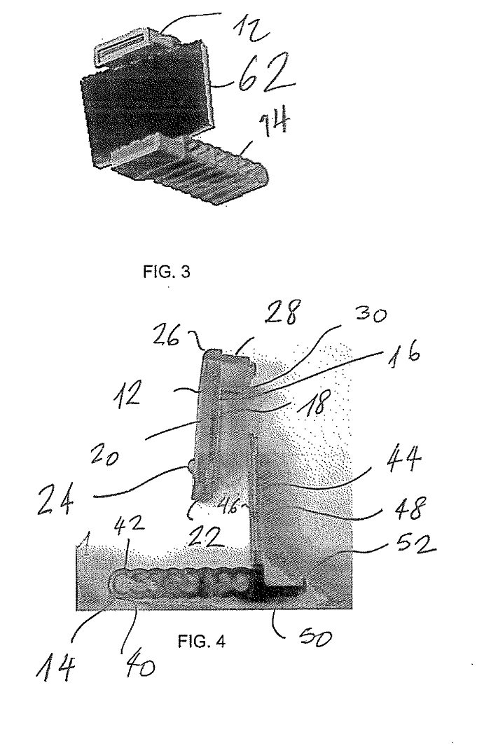

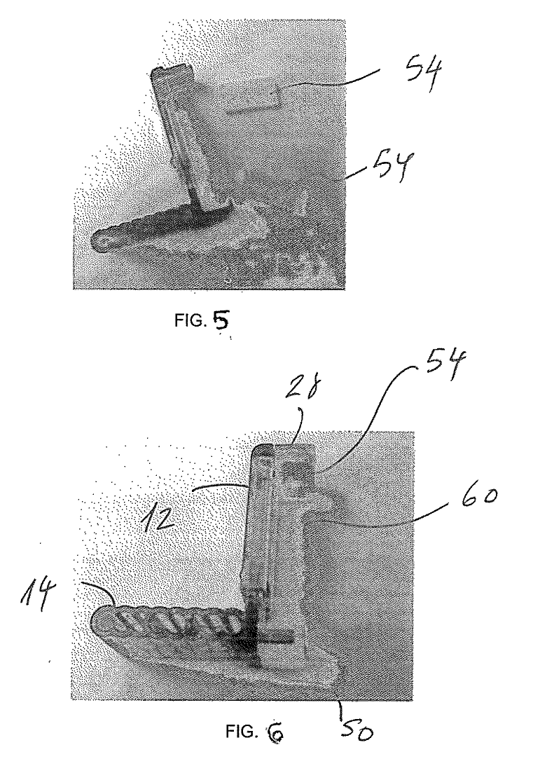

As seen in FIGS. 3, 4, 14 and 15, member 14 includes a base 40 formed with a plurality of transversal apertures 42. These apertures are engaged by a guide assembly as discussed below and illustrated in FIGS. 7 and 8. At one end of the base 40 there is provided an upright element 44 having an inner surface 46 and an outer surface 48. The member 14 further includes a horizontal extension 50 with an upturned lip 52. Member 14 is also provided with two pads made of a resilient material 54 (see FIG. 5). The pads are attached to the extensions 28, 50 respectively.

As seen in FIG. 15, element 44 is formed with a longitudinal aperture 130 having teeth 130 on its lateral sides 132. Element 44 is just wide enough so that it slideably fits in the track formed by rails 112, 114. When the two members 12, 14 are interconnected as shown in FIGS. 3, 16, button 24 passes through the aperture 132 and channel 120, with its teeth 122 intermeshing with the teeth 130. The tongue 120 is flexible so that the button can be pushed inward from surface 18. This motion causes the teeth 122, 130 to separate and allow the two members to move longitudinally with respect to each other, thereby adjusting the distance between the extensions 28 and 50. In one embodiment, the two members can move only if the button 24 is pushed in. Once the button 24 is released, it snaps out to the position shown in FIG. 13, the teeth are intermeshed and the members 12, 14 are locked in position. In another embodiment, the teeth are shaped to allow the members 12, 14 to slide toward each other without pushing the button 24, but the button 24 must still be pushed to increase the distance between extensions 28 and 50.

More particularly, the two members 12, 14 are assembled so that the inner surfaces 18, 46 are facing and are in contact with each other. As described above, the two members 12, 14 are attached to each other in a manner that allows an operator to shift member 12 up or down with respect to member 14 without separating them. The two extensions 28, 50 with their pads 54 and the outer surface 48 form a cavity 60. The vertical spacing between the extensions 28, 50 is changed by moving the members 12, 14 with respect to each other. Therefore a receptor, such as 62 shown in FIG. 3, can be placed into cavity 60 either vertically or horizontally and the spacing between the extensions 28, 50 is adjusted to conform to the respective dimension of the receptor. The pads 54 are made of a somewhat soft material to insure that the receptor 62 is gripped firmly between the extensions 28, 50 without damaging the receptor.

The bite block 10 is connected to an aligning device 70 as shown in FIGS. 7 and 8. The aligning device 70 includes a guide bar 72 having one end inserted into one of the apertures 42. The guide bar 72 supports a positioning ring 74. The ring 74 can be moved up and down the ring 74 and is used to help focus an X-ray beam on the receptor 62.

Given the requirements of a specific X-ray, different shapes and sizes of receptors 62 may be utilized. In addition, these receptors may be placed in position either with the long side disposed vertically or horizontally.

Before the present invention it would be standard practice for the dental technician to have a variety of bite blocks, each block being suited to hold a receptor of a particular type or size. The present invention provides a device with a movable member 12 designed to accommodate receptors of several sizes. The members are interlocked to thereby “lock” the receptor into position by pressing the upper extension down until it “locks” onto the receptor, regardless of the receptor size. Thus the dental technician may complete a full series of exposures without having to switch bite blocks.

When the release button 24 is pressed, the locking mechanism releases the member 12 and the member 12 moves freely, thus releasing the receptor so that the receptor can be re-aligned and or replaced with a different size receptor.

By including apertures along the bite block, the adjustable bite block/sensor holder may be used in conjunction with positioning bars and aiming rings to assure both correct alignment relative to the X-ray source and dentition as well as to avoid “cone cutting” of the image which would result in additional “retakes” and additional radiation exposure to the patient.

Different positioning arms as well as different aiming rings may be used depending on the area of the mount being X-rayed.

The present invention provides for quick and easy assembly of the aiming rings, bite blocks, and indicator arms. In the preferred embodiment, the adjustable bite block/sensor holder may come in three or more sizes and configurations. These include a large size to use with all large size sensors, commonly referred to as sizes 1 and 2, in the anterior and posterior positions, and a smaller version to use with smaller sensors commonly referred to as size 0, also in the anterior and posterior positions, as well as a bite wing assembly that would place the midline of the gripping mechanism at the bite plane of the bite block so as to allow the dentist to take a “bite wing” X-ray, commonly taken in the posterior positions (either bicuspid, premolar or molar regions). In another embodiment of the invention, the bite block is provided in two configurations. One configuration is sized and shaped to accommodate all size 0 receptors. The second configuration is sized and shaped to accommodate all size 1 and 2 receptors.

The present invention, in particular, is effective for providing the optimum position and alignment for dental X-raying, with sensors, of the following regions: Maxillary Incisor, Maxillary Lateral Incisor, Maxillary Cuspid, Maxillary Bicuspid, (Premolar), Maxillary Molar, Mandibular Incisor, Mandibular Cuspid, Mandibular Bicuspid, (Premolar), Maxillary Molar, Mandibular Incisor, Mandibular Cuspid, Mandibular Bicuspid (Premolar), Mandibular Molar, Interproximal Bicuspid (Premolar) and Interproximal Molar.

Based upon the foregoing disclosure, it should now be apparent that the use of the hinged adjustable mechanism/bite block described herein will carry out the objects set forth hereinabove. It is, therefore, to be understood that any variations evident fall within the scope of the claimed invention and thus, the selection of specific component elements or parts can be determined, without departing from the spirit of the invention herein disclosed and described. Numerous modifications may be made to the invention without departing from its scope as defined in the appended claims.

We claim:

1. A dental X-ray positioning device comprising:
a bite block formed of two members arranged and constructed to define a cavity having a variable dimension selected to hold a dental of receptor having one of several dimensions in parallel alignment to an X-ray beam source and a dentition.

2. The device as in claim 1, wherein the bite block includes a release button located on the opposite side of the cavity, said release button releasing said two members to allow said members to be moved with respect to each other to control said variable dimension of said cavity.

3. The device as in claim 1, wherein the locking mechanism is released whenever the release button is pressed, thereby allowing re-positioning of the sensor and/or replacement of the current sensor by another sensor of different size.

4. The device as in claim 1, wherein said bite block is sized and shaped to accept one of size 0 receptors and sizes 1 and 2 receptors.

5. The device as in claim 1, wherein each of said members includes an extension, said extensions cooperating to form soft jaws to securely grip said receptor.

6. The device as in claim 1, wherein said bite block is made from a material capable of sterilization so as to be re-useable following sterilization procedures, so as to eliminate cross-contamination between patients.
